(19) United States
(12) Patent Application Publication
(10) Pub. No.: US 2021/0260522 A1
(43) Pub. Date: Aug. 26, 2021

(54) OXYGEN GENERATOR WITH IMPROVED NOISE AND VIBRATION REDUCTION, COMPACTNESS, AND USER CONVENIENCE

(71) Applicant: KEY NET CO., LTD., Ulsan (KR)

(72) Inventors: Dong-Hyun MOON, Busan (KR); Yi-Jeong YUN, Busan (KR); Jin-Woo LEE, Busan (KR); Byeong-Soo KIM, Changwon-si, Gyeongsangnam-do (KR)

(73) Assignee: KEY NET CO., LTD., Ulsan (KR)

(21) Appl. No.: 16/973,157

(22) PCT Filed: Jul. 3, 2019
(86) PCT No.: PCT/KR2019/008132

§ 371 Date: Dec. 8, 2020

(30) Foreign Application Priority Data
Jun. 11, 2019 (KR) 10-2019-0068854
Jun. 14, 2019 (KR) 10-2019-0070953
Jun. 18, 2019 (KR) 10-2019-0072267
Jun. 18, 2019 (KR) 10-2019-0072270

(51) Int. Cl. B01D 53/22; B01D 63/04; A61M 16/08; A61M 16/06; A61M 16/00; A61M 16/10; C01B 13/02

(52) U.S. Cl. CPC B01D 53/22; B01D 63/046; A61M 16/0875; A61M 16/06; A61M 16/022; C01B 2210/0046; C01B 13/0251; B01D 2053/224; A61M 2202/0208; C01B 2210/001; A61M 16/1005

(57) ABSTRACT

Disclosed is an oxygen generator with improved noise and vibration reduction, compactness, and user convenience. The oxygen generator includes a gas separation membrane module including: gas separation membranes formed of a bundle of hollow fiber membranes; an atmospheric air inlet, to one end of which one end of the gas separation membranes is bonded, sealed, and attached, and the other end of which air in the atmosphere enters; a nitrogen outlet, to one end of which the other end of the gas separation membranes is bonded, sealed, and attached, and the other end of which nitrogen exits; a guide rail where the gas separation membranes are wound in a coil and stored on an inner surface thereof; a gas separation membrane module casing containing the guide rail where the gas separation membranes are stored.

### CROSS-REFERENCE TO PRIOR APPLICATIONS

This application is a National Stage Patent Application of PCT International Patent Application No. PCT/KR2019/008132 (filed on Jul. 3, 2019) under 35 U. S.C. § 371, which claims priority to Korean Patent Application Nos. 10-2019-0068854 (filed on Jun. 11, 2019), 10-2019-0070953 (filed on Jun. 14, 2019), 10-2019-0072267 (filed on Jun. 18, 2019), and 10-2019-0072270 (filed on Jun. 18, 2019), which are all hereby incorporated by reference in their entirety.

### BACKGROUND

The present disclosure relates to an oxygen generator, and more particularly, to an oxygen generator with improved noise and vibration reduction, compactness, and user convenience.

Oxygen generators have been used mainly for medical purposes in hospitals, etc. Recently, there has been growing interest in oxygen generators, geared toward the issues of environmental pollution and the needs of people today who want to live in a clean environment. Notably, oxygen generators are used a lot to help recover from everyday fatigue and activate cells by supplying oxygen to indoor spaces of offices and houses.

There are known methods by which oxygen generators generate oxygen by a chemical reaction, electrolysis, physical separation, etc. In the chemical reaction methods, oxygen is generated by getting metal oxides such as hydrogen peroxide, sodium percarbonate, and alcohol to react with water or by thermally decomposing chemicals such as KMnO4 and KCIO4. In the electrolysis methods, water or water containing an electrolytic substance is split into oxygen and hydrogen.

The physical separation methods include membrane separation, in which gases are separated based on difference in polarity between gases and materials or based on the size of gas molecules, pressure swing adsorption (PSA), in which gases are separated based on the principle of adsorption and desorption of compounds such as a molecular sieve, which is a crystalline solid, and vacuum adsorption (VSA), in which gases are separated under atmospheric pressure and vacuum pressure.

Meanwhile, a gas separation membrane used to separate gases, liquids, or solids, especially specific components such as ion is designed in a way that offers targeting selectivity and allows permeable materials to pass through at low resistance, properly combined with a dense structure or porous structure in order to selectively permeate or filter out specific components.

Notably, hollow fiber membranes are used for air separation through a membrane. Oxygen that has permeated through hollow fiber membranes and nitrogen that has not permeated through them are separated because the hollow fiber membranes have higher permeability for oxygen than for nitrogen.

A gas separation membrane module is mounted to such an oxygen generator. The gas separation membrane module comprises a plurality of gas separation membranes to increase oxygen generating capacity. This requires the gas separation membrane module to be large in size, and, when the gas separation membranes are folded or something, it may break the gas separation membranes or impede the flow of air. Thus, the oxygen generating capacity is not as high as the user wants it to be.

Moreover, a high-performance motor is used for air separation using a gas separation membrane in order to allow atmospheric air to pass through the gas separation membrane at high pressure. A long time use of such a high-performance motor may lead to a decrease in the efficiency of gas separation due to overheating, noise, and vibration of the motor and decrease convenience for users due to noise and vibration.

In addition, with these oxygen generators, a user to be provided with oxygen may find it difficult to notice whether oxygen is being generated or not, since oxygen is a colorless and odorless gas.

Furthermore, users who inhale oxygen through an oxygen generator are mostly elderly people or patients. Oxygen inhalation should be adapted differently for each elderly person or patient depending on their physical characteristics, in order to maximize the effect of oxygen inhalation. However, no discussions have been held yet so far the adaptation of oxygen inhalation for each elderly person's or patient's physical characteristics. There are always concerns about safety incidents since users of oxygen generators are mostly elderly people or patients who live alone.

### SUMMARY

In order to overcome the above-described problems, the present disclosure aims at providing a gas separation membrane module comprising a plurality of gas separation membranes, that is capable of preventing the gas separation membrane module from becoming larger in size, in an oxygen generator with improved noise and vibration reduction, compactness, and user convenience.

Furthermore, the present disclosure aims at providing a gas separation membrane module comprising a plurality of gas separation membranes, that prevents the gas separation membranes from breaking or prevents disturbances in air flow.

Furthermore, the present disclosure aims at providing an oxygen generator comprising such a gas separation membrane module.

Furthermore, the present disclosure aims at providing an oxygen generator capable of significantly reducing possible malfunctions in the oxygen generator due to overheating of a high-performance motor even when the motor is used for a long time when separating gases through gas separation membranes.

Furthermore, the present disclosure aims at providing an oxygen generator capable of significantly reducing the possibility of less convenience for users due to noise and vibration generated from the motor even when the motor is used for a long time, by separating gases through gas separation membranes.

Furthermore, the present disclosure aims at providing an oxygen generator capable of making oxygen inhalation easier by allowing a user to notice the presence of oxygen being generated even though oxygen is colorless and odorless.

Furthermore, the present disclosure aims at providing an oxygen generator for use as a home appliance that can eliminate the concern about user safety incidents by checking the health status of elderly people or patients who live alone and dealing with emergency situations.

Furthermore, the present disclosure aims at providing an oxygen generator that allows each elderly person or each patient to inhale oxygen in different ways depending on their physical characteristics.

Furthermore, the present disclosure aims at providing an oxygen generator that allows elderly people or patients to inhale oxygen easily, even with small lung capacity, taking into consideration that their lung capacity varies with their physical characteristics.

Furthermore, the present disclosure aims at providing an oxygen generator capable of preventing contamination of oxygen inhalation masks when the oxygen generator is used multiple times by an elderly person or patient.

The effects of the present disclosure are not limited to those mentioned above, and other effects that are not mentioned will be clearly understood by those skilled in the art from the following description.

An embodiment of the present disclosure provides a compactible gas separation membrane module for an oxygen generator, the gas separation membrane module comprising: gas separation membranes formed of a bundle of hollow fiber membranes; an atmospheric air inlet, to one end of which one end of the gas separation membranes is bonded, sealed, and attached, and the other end of which air in the atmosphere enters; a nitrogen outlet, to one end of which the other end of the gas separation membranes is bonded, sealed, and attached, and the other end of which nitrogen exits; a guide rail where the gas separation membranes are wound in a coil and stored on an inner surface thereof; and a gas separation membrane module casing containing the guide rail where the gas separation membranes are stored.

The guide rail may be made of an elastic material.

A plurality of through-holes may be formed in an inner surface of the guide rail where the gas separation membranes are stored.

A plurality of projections may be formed on an inner surface of the guide rail where the gas separation membranes are stored.

Another embodiment of the present disclosure provides an oxygen generator comprising the above gas separation membrane module.

Another embodiment of the present disclosure provides an oxygen generator capable of preventing overheating, noise, and vibration of a motor, the oxygen generator comprising an oxygen generator main body internally having a motor and a gas separation membrane module, wherein air compressed or sucked by the motor is separated into oxygen and nitrogen while passing through the gas separation membrane module, and the oxygen and nitrogen are then released through an oxygen release tube and a nitrogen release tube, respectively, and the height or position for nitrogen release inside a motor section is changed as the length of the nitrogen release tube is changed through a sliding guide provided on the nitrogen release tube, so that the nitrogen is released to the motor through the nitrogen release tube to cool the motor.

The sliding guide may comprise a trapping section which traps liquids such as moisture generated when nitrogen passes through the nitrogen release tube.

The nitrogen release tube may comprise an extended outlet section shaped like a trapezoid, whose front portion, middle portion, and rear portion have different curvatures and release angles to generate an acoustic pressure, thus allowing nitrogen air to be released further into the motor section over an extended range.

A heated air outlet section may be provided in an upper part of the motor section and comprise intake portions through which nitrogen air is introduced at a slope after cooling the motor and L-shaped bent outlet openings through which the nitrogen introduced through the intake portions is released.

A collecting plate may be provided in an inside upper part of the motor section, which may be formed as a conductor for collecting scattering impurities and may be negatively (−) charged.

Another embodiment of the present disclosure provides an oxygen generator with improved user safety and convenience, the oxygen generator comprising an oxygen generator main body internally having a motor and a gas separation membrane module, wherein an image projecting means is comprised on one side of the oxygen generator to give the user image guidance about the range for inhalation of oxygen released from the oxygen generator.

The image projecting means may give the user guidance about differences in the concentration of oxygen in the atmosphere, which are caused when oxygen released from the oxygen generator is diluted by mixing with atmospheric air, by making changes to images.

The oxygen generator may recognize the user by interfacing with a user recognition means capable of recognizing the user, and the oxygen generator may have an oxygen outlet for releasing oxygen in four directions so that oxygen is released in the user's direction recognized by the user recognition means, and the image projecting means may project a guidance image in the direction in which oxygen is released.

The oxygen generator may release oxygen when the user recognition means recognizes the user being within the range for oxygen inhalation, and the image projecting means may project a guidance image in the direction in which oxygen is released.

The oxygen generator may increase the volume of oxygen release if the user recognition means recognizes the user being out of the range for oxygen inhalation.

A sensor section may be provided in an upper part of the oxygen generator to recognize the user in four directions parallel to the upper surface of the oxygen generator.

Another embodiment of the present disclosure provides an oxygen generator capable of customizing oxygen inhalation for a user, the oxygen generator comprising: an oxygen generator main body internally having a motor and a gas separation membrane module; a hose portion on one side of the oxygen generator that delivers oxygen released from the oxygen generator to the user; an oxygen inhalation mask provided on one end of the hose portion, which is fitted to the face of the user to inhale oxygen; and an oxygen supply controller provided on one side of the hose portion or oxygen inhalation mask to control the concentration and pressure of oxygen delivered to the user by actuating a fan.

The oxygen supply controller may further comprise outside air intake portions which control communication with the outside atmosphere and induce outside atmospheric air to enter the hose portion or oxygen inhalation mask.

The oxygen supply controller may control the concentration and pressure of oxygen delivered to the user by interfacing with a user recognition means worn by the user.

An anti-contamination means may be provided inside the oxygen inhalation mask and comprise a plurality of anti-contamination films laminated, which the user may remove after use because of contamination concerns.

According to the present disclosure, it is possible to provide a gas separation membrane module comprising a plurality of gas separation membranes, that is capable of preventing the gas separation membrane module from becoming larger in size, in an oxygen generator with improved noise and vibration reduction, compactness, and user convenience.

Furthermore, it is possible to provide a gas separation membrane module comprising a plurality of gas separation membranes, that prevents the gas separation membranes from breaking or prevents disturbances in air flow.

Furthermore, it is possible to provide an oxygen generator comprising such a gas separation membrane module.

Furthermore, it is possible to provide an oxygen generator capable of significantly reducing possible malfunctions in the oxygen generator due to overheating of a high-performance motor even when the motor is used for a long time when separating gases through gas separation membranes.

Furthermore, it is possible to provide an oxygen generator capable of significantly reducing the possibility of less convenience for users due to noise and vibration generated from the motor even when the motor is used for a long time, by separating gases through gas separation membranes.

Furthermore, it is possible to provide an oxygen generator capable of making oxygen inhalation easier by allowing a user to notice the presence of oxygen being generated even though oxygen is colorless and odorless.

Furthermore, it is possible to provide an oxygen generator for use as a home appliance that can eliminate the concern about user safety incidents by checking the health status of elderly people or patients who live alone and dealing with emergency situations.

Furthermore, it is possible to provide an oxygen generator that allows each elderly person or each patient to inhale oxygen in different ways depending on their physical characteristics. Furthermore, it is possible to provide an oxygen generator that allows elderly people or patients to inhale oxygen easily, even with small lung capacity, taking into consideration that their lung capacity varies with their physical characteristics.

Furthermore, it is possible to provide an oxygen generator capable of preventing contamination of oxygen inhalation masks when the oxygen generator is used multiple times by an elderly person or patient.

### BRIEF DESCRIPTION OF THE DRAWINGS

FIG. 1 is a perspective view and structural view of an oxygen generator according to an embodiment of the present disclosure.

FIG. 2 is a reference diagram of a gas separation membrane according to an embodiment of the present disclosure.

FIG. 3 is a configuration view of a gas separation membrane module according to an embodiment of the present disclosure.

FIG. 4 is a cross-sectional diagram of a gas separation membrane module according to an embodiment of the present disclosure.

FIG. 5 is a reference diagram showing how oxygen is released from gas separation membranes according to an embodiment of the present disclosure.

FIG. 6 is an internal structure diagram of an oxygen generator according to an embodiment of the present disclosure.

FIGS. 7 and 8 are reference diagrams of an example of nitrogen release tube provided in an oxygen generator according to an embodiment of the present disclosure.

FIG. 9 is a perspective view of an extended outlet section provided in an oxygen generator according to an embodiment of the present disclosure.

FIG. 10 is a reference diagram showing an example of image projection of an image projecting means according to an embodiment of the present invention.

FIG. 11 is a reference diagram showing an example of how an oxygen generator interfaces upon recognition of a user according to an embodiment of the present disclosure.

FIGS. 12 and 13 are reference diagrams showing a method of user recognition by an oxygen generator according to an embodiment of the present disclosure.

FIG. 14 is a reference diagram showing a user wearing an oxygen inhalation mask of an oxygen generator according to an embodiment of the present disclosure.

FIG. 15 is a cross-sectional diagram showing the configuration and operation of an oxygen supply controller according to an embodiment of the present disclosure.

FIGS. 16 and 17 are reference diagrams showing the configuration and anti-contamination means of an oxygen inhalation mask according to an embodiment of the present disclosure.

### DETAILED DESCRIPTION

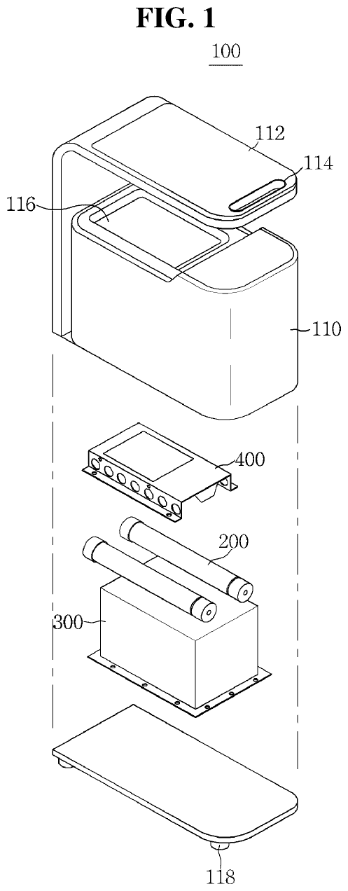

As depicted in FIG. 1, an oxygen generator 100 according to an exemplary embodiment of the present disclosure roughly comprises an oxygen generator main body 110 and a user convenience section 112. The oxygen generator main body 110 internally has a motor 310, a gas separation membrane module 200, a power supply device 400, and a controller (not shown).

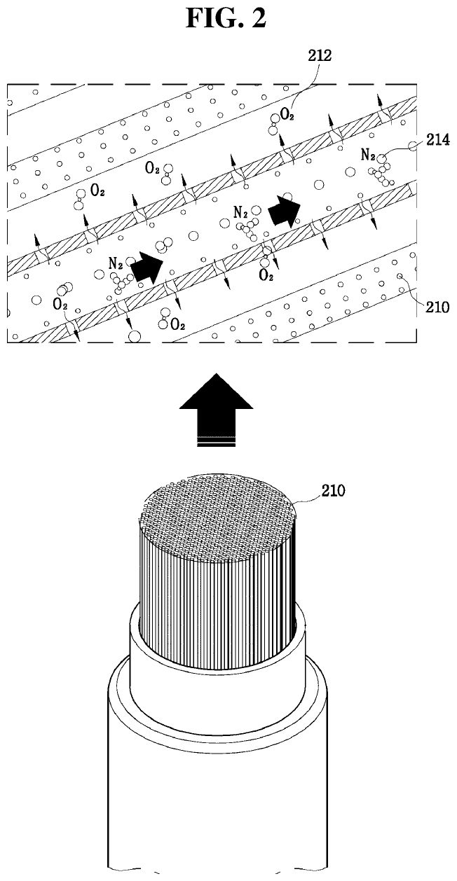

As depicted in FIG. 2, in the gas separation membrane module 200, gases in the atmosphere passing through the inside of hollow fiber membranes 210 are separated into oxygen 212 and other gases than oxygen which are mostly nitrogen 214. The oxygen 212 is supplied to the user, and the nitrogen 214 is vented.

The nitrogen 214 to be vented is released into a motor section (not shown) to cool a motor (not shown) and then vented.

Next, the user convenience section 112 is in the form of a shelf where the user can put a book, a drink, and snacks, and has a handle 114 so that an elderly person or patient who is a primary user of the oxygen generator 100 can stand up holding on to the oxygen generator 100. The handle 114 also may be used for transport so that the user can move the oxygen generator somewhere by grasping the handle 114.

Moreover, a storage space 116 is formed at the top of the oxygen generator main body 110 to store the user's stuff.

Next, an oxygen generator main body support 118 is provided at the bottom of the oxygen generator main body 110. One side of the oxygen generator main body support 118 is constructed of wheels and the other side is constructed of a support bar, so as to prevent safety incidents—such as when the user is pushed away and falls while attempting to stand up holding on to the handle 114—and to move the oxygen generator on the wheels on the one side by lifting the other side.

Next, a user interface (not shown) for allowing the user to control the oxygen generator 100 is provided at the oxygen generator main body 110 or the user convenience section 112 so that the user controls the oxygen generator 100. Through the user interface, the user is given guidance on the amount of oxygen released from the oxygen generator, the operation time, etc.

The oxygen released from the oxygen generator 100 may be delivered to the user via the oxygen generator main body 110, the user convenience section 112, or an oxygen outlet (not shown) provided on one side of the oxygen generator main body 110, or may be delivered to the user via a separate oxygen outlet hose (not shown).

Accordingly, the oxygen generator 100 according to the embodiment of the present disclosure is advantageous in that an elderly person or patient, who is a primary target user, can be supplied with oxygen without any concern for safety incidents and, at the same time, it can be easily used as a home appliance, such as for storing a lot of stuff.

By the way, the gas separation membrane 200 mounted to the oxygen generator 100 to generate oxygen is usually in the form of a long bar, which makes it difficult to manufacture the oxygen generator 100 in a compact size or in an easily portable design.

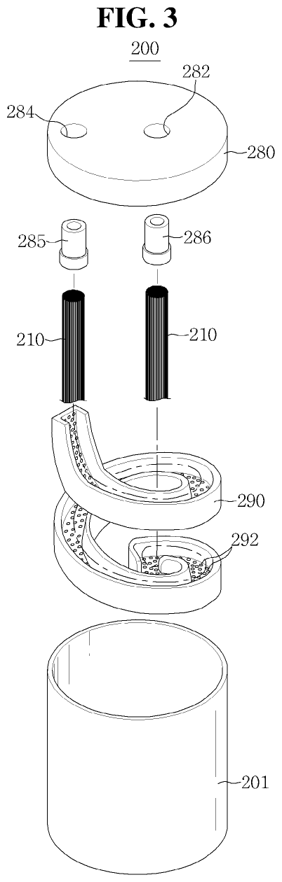

In view of this, as depicted in FIG. 3, the gas separation membrane 200 mounted to the oxygen generator 10 according to the embodiment of the present disclosure is designed in such a manner that the gas separation membranes 210 are wound inside a cylindrical gas separation membrane module casing 201 which is small in length.

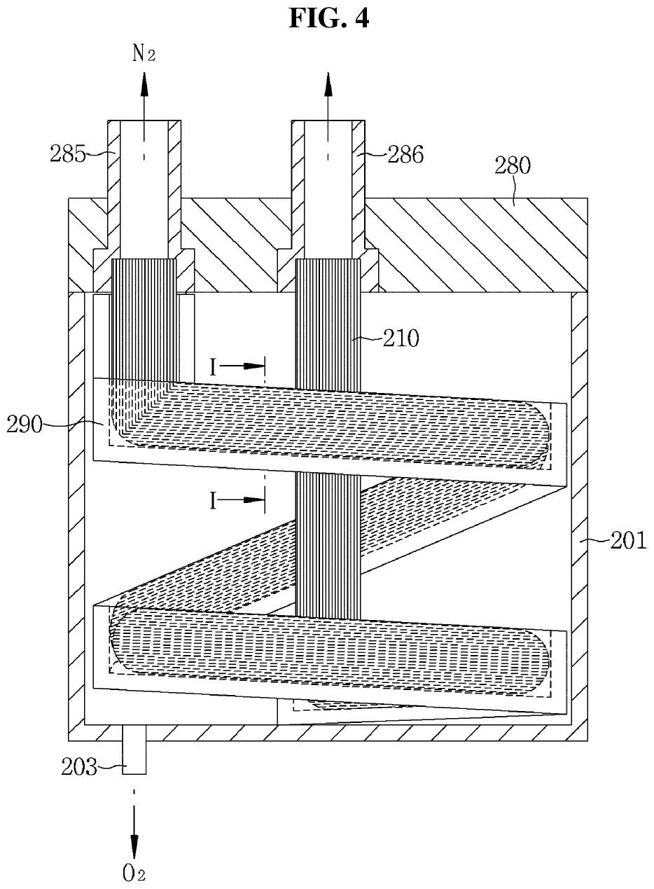

That is, the gas separation membranes 210 are wound and stored inside the gas separation membrane module casing 201 as depicted in FIG. 4. Since the gas separation membranes 210 are stored simply by winding, there may be difficulties in storage, repair, and maintenance, and the gas separation membranes 210 may break or bend at the time of storage by being wound, which may impede the flow of air inside them.

In this regard, in the gas separation membrane module 200 mounted to the oxygen generator 100 according to the embodiment of the present disclosure, the gas separation membranes 210 are stored by using a guide rail 290 where the gas separation membranes 210 can be wound and stored, as depicted in FIGS. 3 and 4.

The guide rail 290 is spiral-shaped and configured such that the gas separation membranes 210 are seated inside, with a sidewall formed on either side to keep the seated gas separation membranes 210 from deviating.

The guide rail 290 may be made of a variety of materials, preferably an elastic material. The gas separation membranes 210 may be compressed when seated in place and stored, in which case the elasticity of the guide rail 290 may prevent damage to the gas separation membranes 210 when compressed.

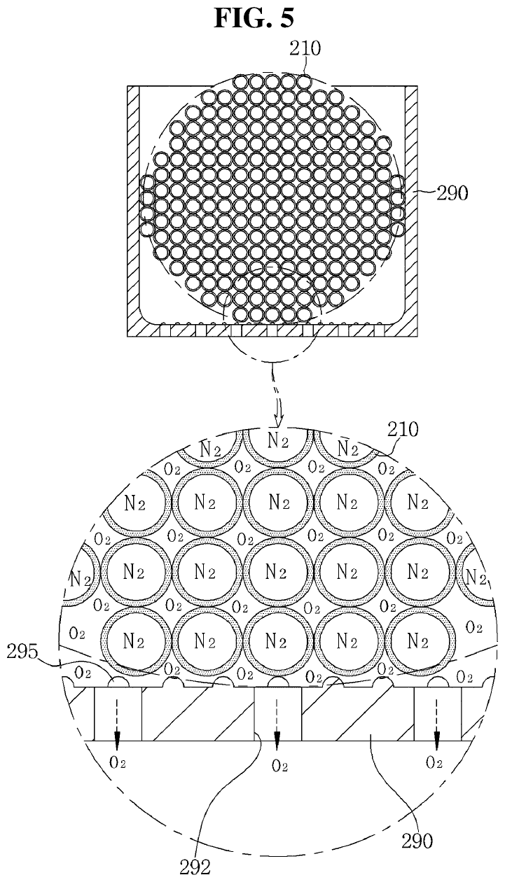

Next, as depicted in FIG. 5, a plurality of pores 292 are formed on the surface where the gas separation membranes 210 of the guide rail 290 are seated, in order to prevent disturbances in the outflow of oxygen when oxygen separated from the gas separation membranes 210 is blocked by the inside surface of the guide rail 290.

That is, as depicted in FIG. 5, oxygen separated from the gas separation membranes 210 is released out of the guide rail 290 via the pores 292.

Next, as depicted in FIG. 4, a plurality of projections 290 are formed on the surface where the gas separation membranes 210 of the guide rail 290 are seated, in order to prevent disturbances in the outflow of oxygen separated from the gas separation membranes 210 when the gas separation membranes 210 and the inside surface of the guide rail 290 are firmly attached together.

That is, as depicted in FIG. 5, the projections 295 allow for the formation of space between the guide rail 290 and the gas separation membranes 210 seated in the guide rail 290, thus allowing oxygen to be smoothly released via the pores 292.

With this configuration of the gas separation membrane module 200 according to the embodiment of the present disclosure, air enters through an atmospheric air inlet 285 and passes through the gas separation membranes 210, and oxygen is supplied to the user through an oxygen generating vent 203 and what's left in the air after the separation of oxygen, which is mostly nitrogen, is supplied to the motor section through a nitrogen outlet 286 and used to cool the motor section.

The gas separation membrane module 200 according to the embodiment of the present disclosure facilities the ventilation of oxygen, once released into the gas separation membrane module casing 201, through the oxygen generating vent 203 by the vertical movement of the guide rail 290. The guide rail 290 may be moved vertically by a vertical sliding means using a motor which is provided at the atmospheric air inlet 285 and the nitrogen outlet 286.

With this configuration, the gas separation membrane module 200 according to the embodiment of the present disclosure may reduce the size and volume of the gas separation membrane module 200, prevent damage to the gas separation membranes 210 at the time of storage, and provide users more convenience in storing the gas separation membranes 210.

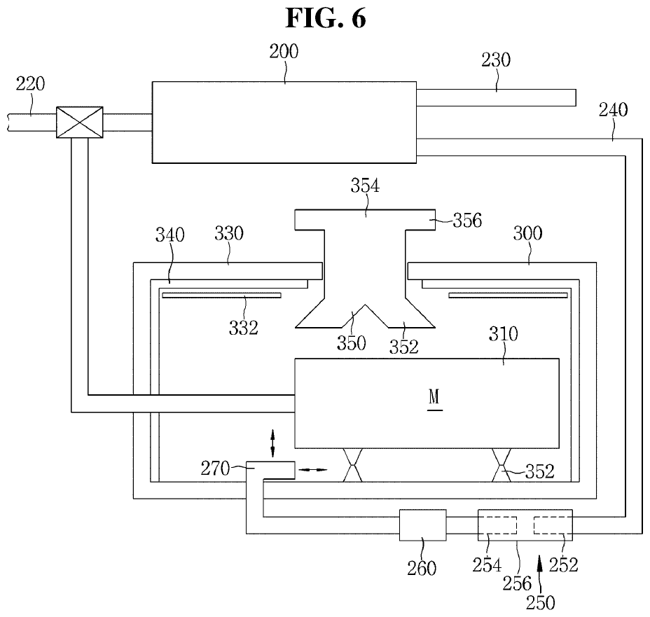

Hereinafter, the overheating, noise, and vibration reduction of the oxygen generator 100 according to the embodiment of the present disclosure will be described with reference to FIG. 6. As depicted in FIG. 6, in the oxygen generator 100 according to the embodiment of the present disclosure, air compressed by the motor 310 is separated into oxygen 212, nitrogen 214, etc. as it passes through the gas separation membrane module 200, and the oxygen 212 and the nitrogen 214 and other gases are then released through an oxygen release tube 230 and a nitrogen release tube 240, respectively.

The oxygen 212 is released to the user through the oxygen release tube 230, and the nitrogen 214 and other gases are supplied into the motor section 300 through the nitrogen release tube 240 to cool the motor 310 and then released.

Although the above description has been made on the assumption that the motor 310 compresses atmospheric air from the front of the gas separation membrane module 200 and sends the compressed atmospheric air to the gas separation membrane module 200, the user may choose to configure the motor 310 in such a manner as to suck atmospheric air from the rear of the gas separation membrane module 200 into the gas separation membrane module 200. Also, atmospheric air may be sucked from the oxygen release tube 230 and the nitrogen release tube 240 respectively by using two small motors.

As depicted in FIG. 6, since the motor section 300 compresses atmospheric air at high pressure and then sends it to the gas separation membrane module 200, the motor 310 is provided inside a motor casing 330 made of thick metal, more specifically, above a vibration damper 320 inside the motor casing 330.

Also, an acoustic absorbent 340 is provided inside the motor casing 330 to reduce noise generated from the motor 310.

Thus, in the motor section 300 according to the embodiment of the present disclosure, the motor 310 is cooled by using nitrogen released through the nitrogen release tube 240, in order to prevent overheating caused by the heat in the motor 310 that is not released due to the motor casing 310 and the acoustic absorbent 340.

At this point, the oxygen generator 100 according to the embodiment of the present disclosure does not simply cool the motor 310 by using nitrogen released through the nitrogen release tube 240, but also greatly enhances the noise and vibration reduction effect and the cooling effect by including a sliding section 250, a cooling section 260, and an extended outlet section 270, in addition to the nitrogen release tube 240, as depicted in FIG. 6.

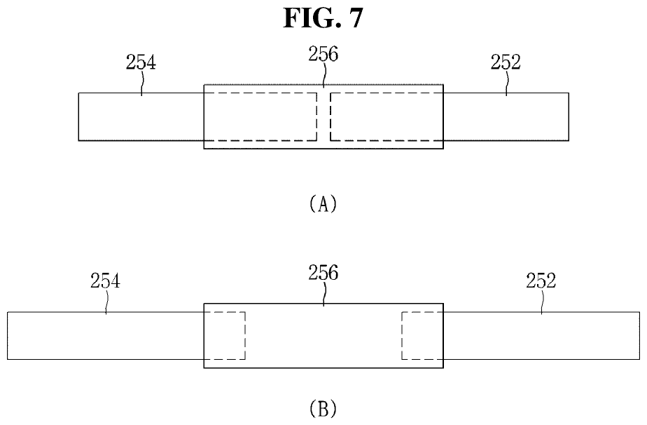

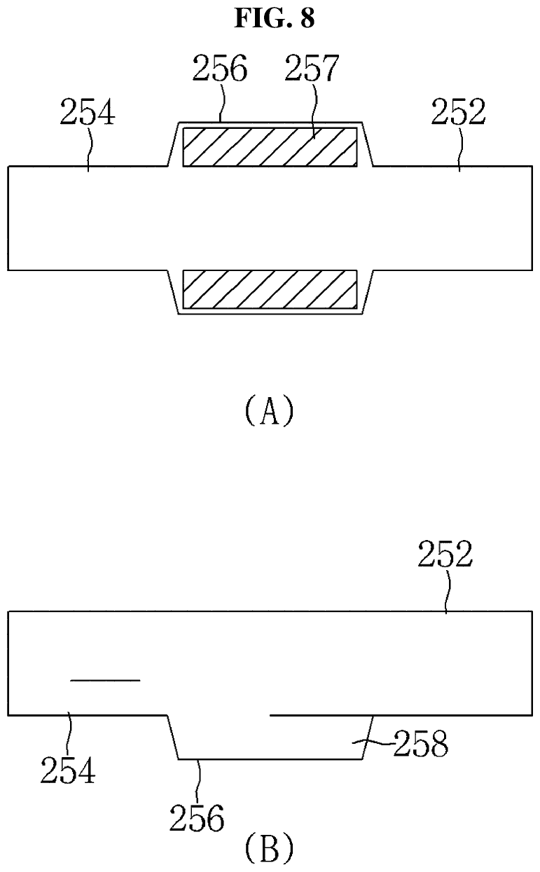

Firstly, as for the sliding section 250 as depicted in FIG. 7, a front nitrogen release tube 254 and a rear nitrogen release tube 252 are connected by a sliding guide 256, and the front nitrogen release tube 254 and the rear nitrogen release tube 252 are configured to slide laterally by means of the sliding guide 256.

With this configuration, the front nitrogen release tube 254, which may be made of an elastic material, may release nitrogen at varying heights and positions inside the motor section 300, as indicated by the arrows at the bottom of FIG. 6, so that the height or position for nitrogen release may be changed depending on the performance and configuration of the motor 310, thereby enhancing the cooling efficiency.

Alternatively, front nitrogen release tubes 254 having different heights and positions for releasing may be provided, so that the height or position for nitrogen release may be changed depending on the performance and configuration of the motor 310, thereby enhancing the cooling efficiency.

That is, given the fact that the size and configuration of the motor 310 and the height and size of the damper 320 may be changed depending on the use and capacity of the oxygen generator 100, the oxygen generator 100 can be designed to be easier to install and manufactured at much lower costs, merely by including front nitrogen release tubes 254 with different configurations. Next, noise may be reduced by adding an acoustic absorbent 257 to the sliding guide 256 between the front nitrogen release tube 254 and the rear nitrogen release tube 252, as depicted in (A) of FIG. 7, or liquids such as moisture generated when nitrogen passes through the nitrogen release tube 240 may be trapped by a trapping section 258 as depicted in (B) of FIG. 7.

Next, a cooling section 260 for additionally cooling nitrogen may be provided on the front nitrogen release tube 254. The cooling section 260 may be implemented in the form of a cooling section using a thermoelement.

Next, an outlet section for releasing nitrogen air into the motor section 300 is provided at the tip of the front nitrogen release tube 254. In the oxygen generator 100 according to the embodiment of the present disclosure, the outlet section comes in the form of an extended outlet section 270.

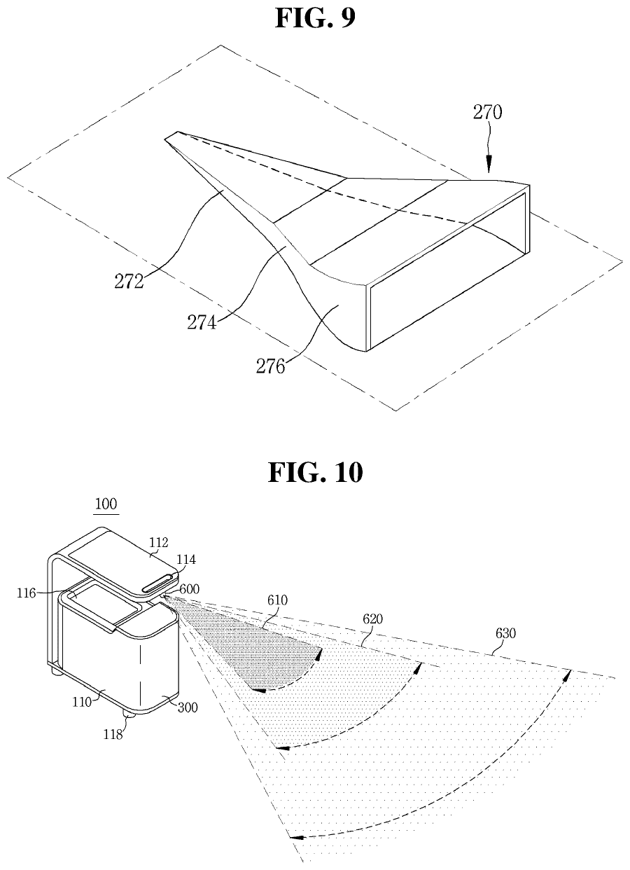

The extended outlet section 270 is shaped like a trapezoid, as depicted in FIG. 9, and its front portion 272, middle portion 274, and rear portion 276 have different curvatures and release angles. With the different curvatures and release angles, an acoustic pressure is generated inside the extended outlet section 270, thus allowing cooled nitrogen air to be released further into the motor section 300 over an extended range.

As such, the cooling efficiency inside the motor section 300 may be further enhanced. That is, nitrogen is an inert gas, meaning that a nitrogen environment is maintained throughout the inside of the motor section 300, thus allowing for efficient cooling and improving the operation stability of the motor.

Next, a heated air outlet section 350 is provided in an upper part of the motor section 300 according to the embodiment of the present disclosure. The heated air outlet section 350 has sloped intake portions 352 inside the motor section 300 and L-shaped bent outlet openings 356 outside the motor section 300.

Accordingly, with this configuration, it is possible to prevent noise from the motor 310 inside the motor section 300 as much as possible from being transmitted directly to the outside through the heated air outlet section 350 connected to the outside.

That is, noise from the motor 310 is reflected and transmitted through the sloped intake portions 352 and the L-shaped bent outlet openings 35, thereby reducing the amount of noise transmitted to the outside.

Next, a collecting plate 332 is provided in an inside upper part of the motor section 300. The collecting plate 332 is formed as a conductor on an upper inner wall of the inside of the motor section 300 in order to collect scattering impurities and prevent damage to the motor due to the impurities. The collecting plate 332 is negatively (−) charged.

With this configuration, the present disclosure offers the advantage of reducing noise and vibration in the oxygen generator 100 and reducing malfunctions in the oxygen generator 100 due to overheating of the motor 310.

However, the user of the oxygen generator 100 may not be able to easily notice whether and how much oxygen is being generated, since oxygen is a colorless and odorless gas, even when oxygen is released through the oxygen outlet provided on one side of the user convenience section 112 or oxygen generator main body 110.

In view of this, according to an exemplary embodiment of the present disclosure, the oxygen generator 100 helps the user easily check whether oxygen is being released and inhale oxygen by showing an image to the user when oxygen is released through the oxygen outlet provided on one side of the user convenience section 112 or oxygen generator main body 110 as depicted in FIG. 1.

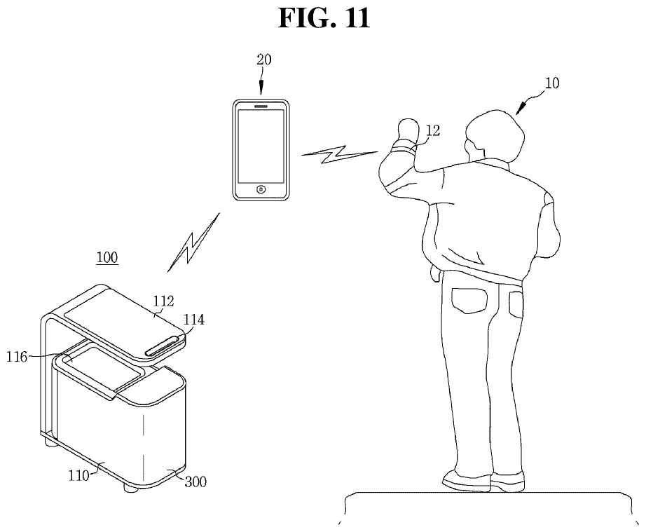

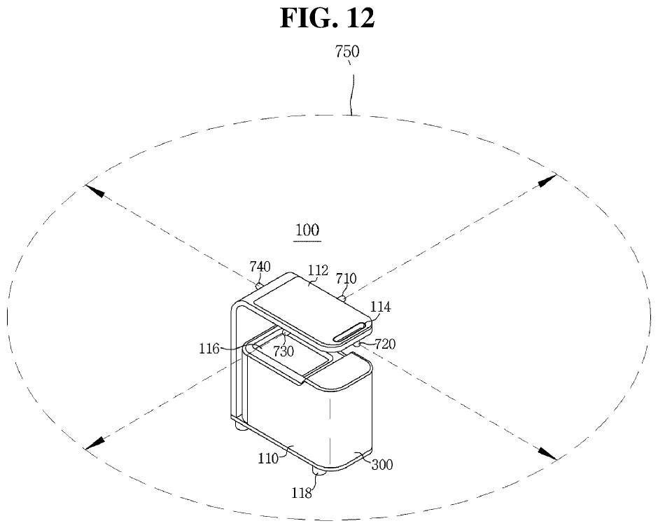

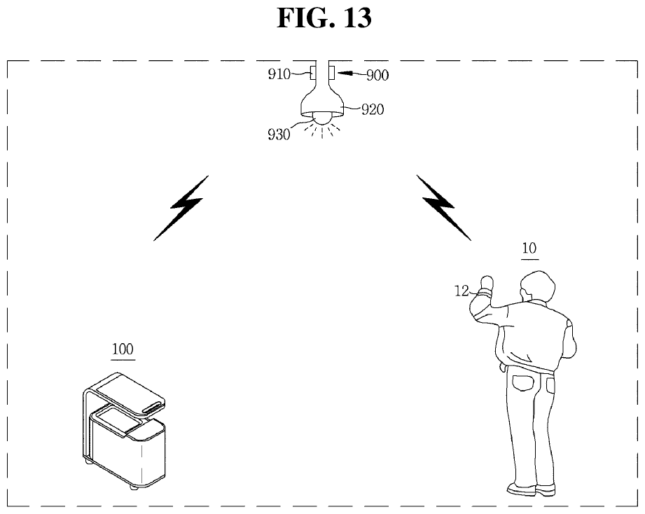

That is, as depicted in FIG. 10, the direction in which oxygen is released from the oxygen outlet and the volume of oxygen release are indicated through an image projecting means 600 provided on one end of the user convenience section 112, thereby enhancing the effect of oxygen inhalation by the user.

The image projecting means 600 may be implemented as a means capable of emitting a variety of lights such as lasers, LEDs, etc., and indicates the range of oxygen release on the floor where the oxygen generator 100 is placed.

Since oxygen released from the oxygen generator 100 is released to the atmosphere, the oxygen mixes with the atmosphere, once beyond a certain range, and the concentration of oxygen becomes similar to that in the atmosphere.

Therefore, in order for the user to inhale oxygen released from the oxygen generator 100 within a range where they are expected to improve their health status, it is desirable that they inhale within a certain range from the direction in which oxygen is released from the oxygen generator 100.

In view of this, according to an exemplary embodiment of the present disclosure, the oxygen generator 100 gives guidance through the image projecting means 600, about the range for oxygen inhalation where the user can improve their health status.

Moreover, as depicted in FIG. 10, the user is given guidance about the range for oxygen inhalation where they can inhale oxygen effectively, by gradually changing the color and intensity of images 610, 620, and 630.

Furthermore, the user is able to easily notice whether oxygen is being generated, through the image projecting means 600.

In addition, as depicted in FIG. 11, the oxygen generator 100 according to the embodiment of the present disclosure is configured in such a manner that the user can be recognized through a mobile device 20 such as a cellular phone used by the user 10 or a user recognition means 12 worn by the user.

The user recognition means 12 is configured to be worn by the user's body and recognize the user's physical information such as the user's blood pressure, oxygen saturation, body temperature, etc.

Also, the user's mobile device 20 or the user recognition means 12 may be detected by a sensor (not shown) provided in the oxygen generator 100, and the two are connected over a network.

Accordingly, as depicted in FIG. 11, the oxygen generator 100 may generate oxygen by detecting access from the user, increase the volume of oxygen release if the distance from the user is out of an effective range for oxygen inhalation so that the user is within the effective range for oxygen inhalation, or increase the effect of oxygen inhalation by the user by giving image guidance through the image projecting means 600 so that the user is within the effective range for oxygen inhalation.

The oxygen generator 100 according to the embodiment of the present disclosure may have the oxygen outlet in four directions of the oxygen generator main body 110 or user convenience section 112, and may be controlled such that oxygen is released from the oxygen outlet positioned in the user's direction depending on the user's position.

The oxygen generator 100 may be controlled to restrict the operation of the image projecting means 600 or minimize the amount of light radiation so as not to disturb the user's sleep at night.

Hereinafter, the functions of the oxygen generator 100 according to the embodiment of the present disclosure that recognize user emergency situations and deal with safety incidents involving these situations will be described in details.

The oxygen generator 100 according to the embodiment of the present disclosure comprise a sensor section 700 on the oxygen generator main body 110 or user convenience section 112 which uses laser, ultrasonic waves, infrared rays, etc. to recognize an object.

The oxygen generator 100 may have the sensor section 700 in four directions 710, 720, 730, and 740 on one side of the oxygen generator main body 110 or user convenience section 112. Preferably, the sensor section may be configured to detect an object only linearly, as depicted in

FIG. 10.

That is, the oxygen generator 100 has a height of around 1 m, which usually reaches from the user's knees to waist. With this height, when the user is sitting or move around the room or living room where the oxygen generator 100 is set up, the user may be recognized as long as linear object detection is possible.

However, in a case where the user collapses suddenly while the user is being recognized, the user is likely to be lying in the room or living room, which makes it impossible to linearly recognize the user.

Thus, if the user being recognized suddenly becomes unrecognizable, rather than becoming farther and farther from view and no longer recognizable, the oxygen generator 100 decides that the user is in an emergency situation, and sends an emergency rescue alert to an emergency contact or a guardian of the patient to deal with the user's emergency situation.

Alternatively, when the user recognition means 12 in inactive state is activated and measures the user's physical information such as blood pressure and pulse rate for the second time and then recognizes that the user is in an emergency situation, it sends an emergency rescue alert to an emergency contact or a guardian of the patient to deal with the user's emergency situation.

Next, as depicted in FIG. 13, the oxygen generator 100 according to the embodiment of the present disclosure is configured to interface with an IoT sensor 910 that can be fitted to a light fixture 920 on the ceiling of the room, so as to recognize a user emergency situation.

That is, the IoT sensor 910, fitted to the light fixture 920, requires no power supply means and is capable of recognizing the user for 24 hours by using the power supplied to the light fixture 920, and also allows for user recognition from a height without interference with other electronics or furniture since it is fitted to a ceiling.

Therefore, the IoT sensor 910 may be fitted to a light fixture 920 at the entrance of a house or in a room or living room and capable of recognizing the user for 24 hours, and may notify the oxygen generator 100 in the event of an emergency situation and send an emergency rescue alert to an emergency contact or a guardian of the patient to deal with the user's emergency situation.

For example, let's say that an IoT sensor 910 fitted to a light fixture or porch light at the entrance of a house recognized the user going into the house but has not detected the user stepping out of the house for a certain period of time, it may decide that an emergency situation might have occurred. Also, assuming that the user went to a bathroom but has not come out after a certain period of time, the IoT sensor 910 may decide that an emergency situation might have occurred.

With this configuration, the present disclosure may enhance the effect of oxygen inhalation by the user from the oxygen generator 100 and prevent safety incidents and conduct a rescue operation.

However, it may be difficult for a user 10 with small lung capacity to take better advantage of the effect of oxygen inhalation when inhaling oxygen using the oxygen generator 100, because the user 10 is simply connected to the oxygen generator 100 via a hose through which oxygen is delivered.

In view of this, the oxygen generator 100 according to the embodiment of the present disclosure comprises an oxygen inhalation mask section 400 by which even a user 10 in a physically weak condition can inhale oxygen easily.

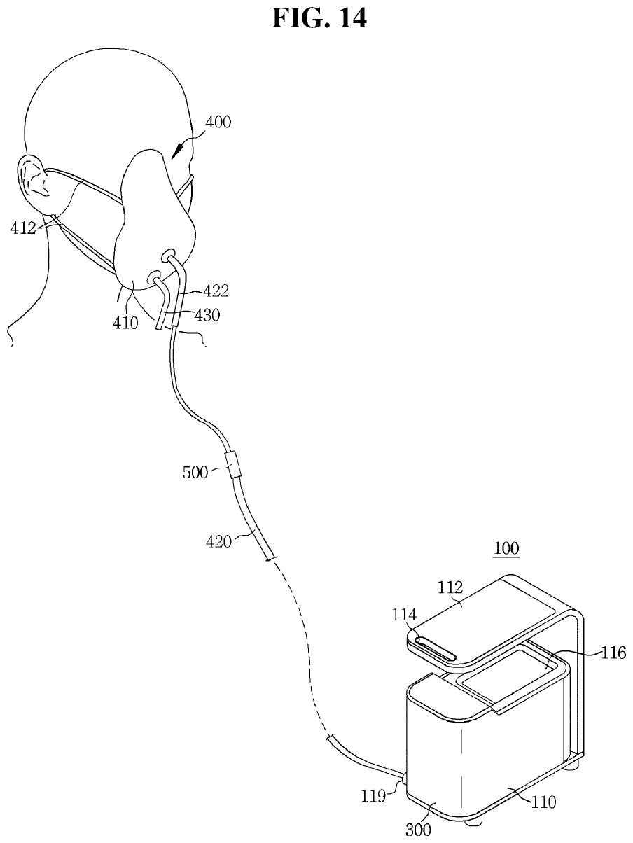

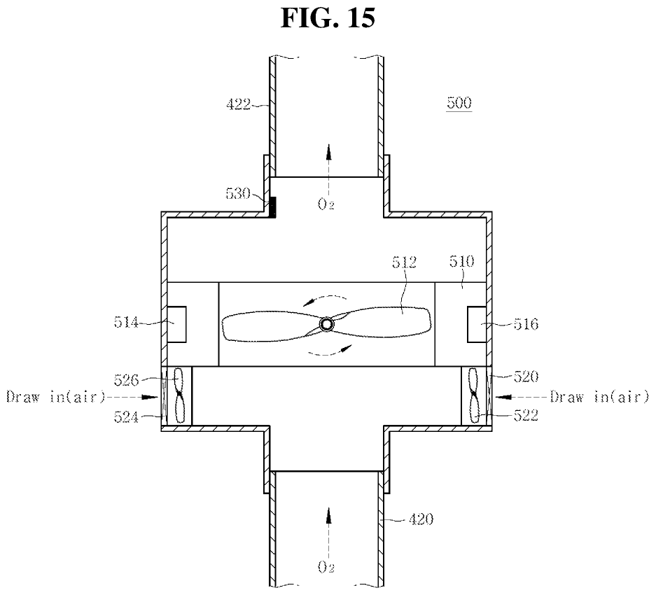

As depicted in FIG. 14, the oxygen inhalation mask section 400 comprises a hose portion 420 that is connected to the oxygen generator main body 110 and delivers oxygen generated in the oxygen generator main body 110 to the user.

One end of the hose portion 420 is connected to the oxygen generator main body 110, and the other end is connected to the oxygen inhalation mask 410 so as to make it easy for the user to inhale oxygen.

Also, an oxygen supply controller 500 is provided in the middle of the hose portion 420 to regulate the amount of oxygen supply or the like depending on the user's physical characteristics.

Moreover, the oxygen inhalation mask 410 may be worn by users of different body shapes. In this embodiment, the oxygen inhalation mask 410 is configured in such a way that the exterior made of a plastic or elastic material and the nose and mouth of the user do not communicate.

This is because, if the oxygen inhalation mask 410 is made of a material such as cotton, air may enter the mask, which may lead to a decrease in oxygen inhalation efficiency.

The user may have the oxygen inhalation mask 410 tightly secured to the face to cover their mouth and noise by looping a cradle 412 of the oxygen inhalation mask 410 around the ears or head.

Furthermore, the oxygen inhalation mask 410 may comprise an exhalation vent 430 for venting the user's exhaled breath.

Thus, the user may inhale oxygen through an oxygen intake port 422 and then exhale their breath through the exhalation vent 430, thereby further increasing the effect of oxygen inhalation by the user.

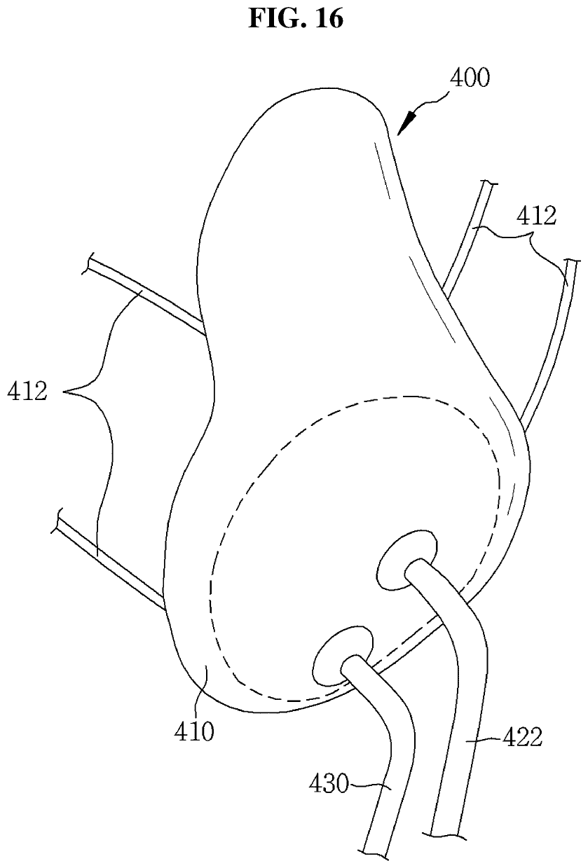

Next, the oxygen supply controller 500 provided in the oxygen inhalation mask 410 according to the embodiment of the present disclosure will be described in detail with reference to FIG. 15.

The oxygen supply controller 500 allows elderly people or patients with small lung capacity to inhale oxygen easily depending on the user's physical characteristics.

That is, as depicted in FIG. 15, an oxygen intake fan section 510 is provided inside the oxygen supply controller 500.

An oxygen intake fan 512 is provided in the oxygen intake fan section 510. The fan is operated to draw in oxygen and release it to the oxygen intake port 422 so that even an elderly person or patient with small lung capacity can easily inhale oxygen.

Also, outside air intake portions 520 and 524 are provided in the oxygen supply controller 500. The outside air intake portions 520 and 524 have an on-off switch (not shown) on the outer side which can be opened and closed, so that the on-off switch is opened to draw in outside air when necessary. The on-off switch may be embodied in various ways depending on the configuration chosen by the user, including a sliding on-off switch with a through-hole formed in it.

As for the outside air intake portions 520 and 524, if the user only inhales a large amount of oxygen for a long time, it may have rather negative effects on the user. Thus, some outside air may be mixed in with oxygen inhaled by the user and admitted toward the oxygen intake port 422. That is, for an initial period of time, the user is supposed to only inhale a high concentration of oxygen depending on the user's physical characteristics, and, over time, the outside air intake portions 520 and 524 are partially opened and closed and the outside air intake portion fans 522 and 526 are actuated to decrease the concentration of oxygen.

Accordingly, the oxygen generator 100 according to the embodiment of the present disclosure offers the advantage of allowing even a user in a physically weak condition, such as low lung capacity, to inhale oxygen easily.

Moreover, as depicted in FIG. 11, the oxygen generator 100 may interface with the user recognition means 12 which is worn by the user to measure the user's physical information such as the user's blood pressure, oxygen saturation, body temperature, etc., and may be controlled such that the user inhales oxygen depending on their physical characteristics.

In addition, as depicted in FIG. 15, a sensor 530 may be fitted to the oxygen supply controller 500 to control it.

That is, the sensor 530 may include a flow measurement sensor, an oxygen concentration measurement sensor, etc., and the oxygen supply controller 500 may be controlled based on measurements made by the sensor 530.

The above control operation may be performed freely as the user chooses, such as by a preset program or a program that takes information on the user's physical characteristics through a mobile device 20 and provides it in real time.

Accordingly, the oxygen generator 100 according to the embodiment of the present disclosure allows the user to inhale oxygen easily depending on the user's physical characteristics and further enhances the effect of oxygen inhalation by the user.

Next, the oxygen inhalation mask 410 of the oxygen generator 100 according to the embodiment of the present disclosure comprises an anti-contamination means 440 for preventing contamination of the oxygen inhalation mask 410 and the user.

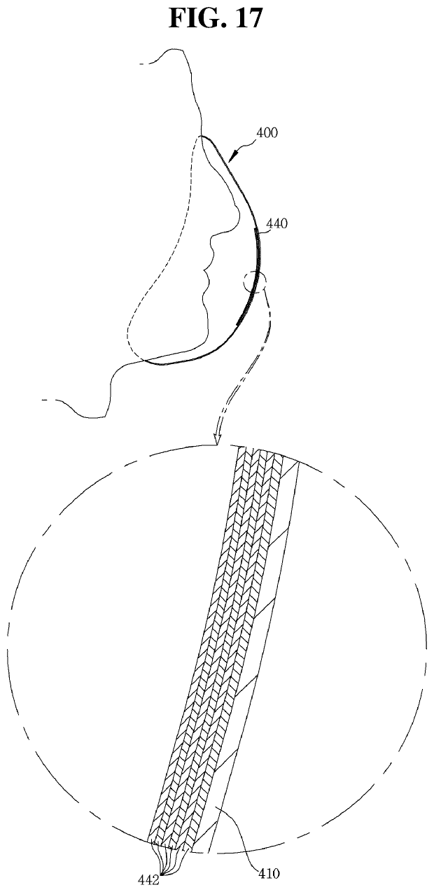

As depicted in FIG. 17, the anti-contamination means 440 may be provided in such a way that a plurality of anti-contamination films 442 are laminated.

That is, the user may inhale oxygen easily without contamination of the oxygen inhalation mask 410 and a resulting infection in the user and also without having to wash it, by removing the top anti-contamination film 442 once the oxygen inhalation mask 410 has been worn for a certain length of time and then removing the next anti-contamination film 442 after the oxygen inhalation mask 410 has been re-worn for the same length of time.

When all of the anti-contamination films 442 of the anti-contamination means 440 have been used, a new anti-contamination means 440 may be fitted to the oxygen inhalation mask 410 and used.

Moreover, the anti-contamination films 442 may have different colors so that the user can easily decide whether to replace them. That is, the films may be designated as Week 1, Week 2, and Week 3, and the film for Week 1 may be designated as yellow, the film for Week 2 as blue, and the film for Week 3 as green. This way, the user may easily replace the films.

With this configuration, the oxygen generator 100 according to the embodiment of the present disclosure may further enhance convenience for users.

While the exemplary embodiments of the present disclosure are provided as described above, it is obvious that various changes, modifications, and equivalents thereof may be used, and that the above exemplary embodiments may be suitably modified and equally applied. Therefore, the above descriptions do not limit the scope of the present disclosure, which is defined by the limitations of the following claims.

An oxygen generator with improved noise and vibration reduction, compactness, and user convenience according to the present disclosure may be effectively used for air purifiers, medical oxygen generators, etc.

1-5. (canceled)

6. An oxygen generator capable of preventing overheating, noise, and vibration of a motor, the oxygen generator comprising an oxygen generator main body internally having a motor and a gas separation membrane module,
wherein air compressed or sucked by the motor is separated into oxygen and nitrogen while passing through the gas separation membrane module, and the oxygen and nitrogen are then released through an oxygen release tube and a nitrogen release tube, respectively, and
the height or position for nitrogen release inside a motor section is changed as the length of the nitrogen release tube is changed through a sliding guide provided on the nitrogen release tube, so that the nitrogen is released to the motor through the nitrogen release tube to cool the motor.

7. An oxygen generator with improved user safety and convenience, the oxygen generator comprising an oxygen generator main body internally having a motor and a gas separation membrane module,
wherein an image projecting means is comprised on one side of the oxygen generator to give the user image guidance about the range for inhalation of oxygen released from the oxygen generator.

8. The oxygen generator of claim 7, wherein the image projecting means gives the user guidance about differences in the concentration of oxygen in the atmosphere, which are caused when oxygen released from the oxygen generator is diluted by mixing with atmospheric air, by making changes to images.

9. The oxygen generator of claim 7, wherein the oxygen generator recognizes the user by interfacing with a user recognition means capable of recognizing the user, and
the oxygen generator has an oxygen outlet for releasing oxygen in four directions so that oxygen is released in the user's direction recognized by the user recognition means, and the image projecting means projects a guidance image in the direction in which oxygen is released.

10. An oxygen generator capable of customizing oxygen inhalation for a user, the oxygen generator comprising:
an oxygen generator main body internally having a motor and a gas separation membrane module;
a hose portion on one side of the oxygen generator that delivers oxygen released from the oxygen generator to the user;
an oxygen inhalation mask provided on one end of the hose portion, which is fitted to the face of the user to inhale oxygen; and
an oxygen supply controller provided on one side of the hose portion or oxygen inhalation mask to control the concentration and pressure of oxygen delivered to the user by actuating a fan.

11. The oxygen generator of claim 10, wherein the oxygen supply controller further comprises outside air intake portions and which control communication with the outside atmosphere and induce outside atmospheric air to enter the hose portion or oxygen inhalation mask.

12. The oxygen generator of claim 10, wherein the oxygen supply controller controls the concentration and pressure of oxygen delivered to the user by interfacing with a user recognition means worn by the user.
